(19) United States
(12) Patent Application Publication
(10) Pub. No.: US 2010/0010540 A1
(43) Pub. Date: Jan. 14, 2010

(54) Device for vertebral stabilization

(76) Inventor: Gi-Hoon Park, Gimhae-Si (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/232,626

(22) Filed: Sep. 22, 2008

(30) Foreign Application Priority Data
Jul. 9, 2008 (KR) 10-2008-0066507

(51) Int. Cl. A61B 17/70; A61B 17/04

(52) U.S. Cl. 606/246; 606/264; 606/301; 606/278

(57) ABSTRACT

A device for fixing vertebral has developed that is comprising: a constrainer having a constraint base and a constraint mount forming two lateral cutouts for receiving a connecting rod; an anchor having a screw for installing into the vertebral, a stem to be inserted into the constrainer, and an annular ring; a pivot having an insertion hole, a plurality of slits extended from a mouth of insertion hole, a snap-fitting groove forming on the inner peripheral surface for snapping-fit to the annular ring: and a constraint nut having a male thread formed on outer peripheral surface, and a hexagonal recess on the head for rotating by a tool. When the constraint nut is rotated clockwise, the constraint members and connecting rod are depressed against the peripheral seat to be immobile. But, the counterclockwise rotation of constraint nut makes the constraint members releasing and the pivot is free to swivel with respect to the anchor.

### BACKGROUND OF THE INVENTION

1. Field of the Invention

The present invention relates to a vertebral fixing device, and more particularly, the vertebral stabilizer has adapted a pivot to freely swivel with respect to the anchor and a constrainer to fix at a proper location, so that it can be easily positioned, adjusted and tightly coupled to a connecting rod. The free swiveling of the pivot and constrainer can solve the problem of the limited anchor diameter for installing into a vertebra, and avoid the problem of the bulky head of anchor.

2. Description of the Prior Art

As generally known in the surgical technology, the degenerative vertebral diseases including the disk degenerative diseases (DDD), spinal stenosis and spondylolisthesis are subjected to conservative treatment first, and if no improvement occurs, surgical treatment follows. The surgical treatment consists of decompression, fusion (ALIF, PLIF, or posterolateral fusion), and fixation for fixing vertebrae. In some cases, the surgical procedure consists of decompression only, or both decompression and fusion. However, the surgery in most cases finishes with fixation.

The fixation employs a vertebral fixing device having a connection rod and a cross-link, such as a pedicle screw. The vertebral fixing device includes a screw-type fixing anchor installed into the pedicle of a vertebra or a sacrum to immobilize a damaged spinal portion after restoring it to a normal condition, and fastening members for coupling anchors to the connecting rod.

FIG. 1 is a perspective view of a conventional vertebral fixing device. As shown, the conventional vertebral fixing device includes an anchor 12 and anchor head 13 inserted into a constrainer 11 and fixed to a vertebra with a retainer member 21 for coupling the anchor 12 and the anchor head 13 to a connecting rod 23.

According to this construction, the anchor body 12 is first moved through the constrainer 11 so that the anchor head 13 engages with the constrainer 11. The anchor body 12 is then installed into and fixed to a vertebra.

Thereafter, the connecting rod 23 is coupled to the constrainer 11 crosswise. The fact that the constrainer 11 can roll and tilt with regard to the anchor head 13 in this condition facilitates the coupling process. The constraint member 21 is then threaded onto the constrainer 11 to compress and fix the connecting rod 23 and the anchor head 13.

Although the conventional vertebral fixing device has a comparatively simple structure using a rotating ball-type anchor head 13, it has an advantage that the constrainer 11 can be moved freely during surgery. In other words, the constrainer 11 and the connecting rod 23 can be easily coupled to each other during surgery.

However, the conventional vertebral fixing device has a serious problem resulting from the structural requirement that the rotatable ball-type anchor head 13 must engage with the constrainer 11 while the anchor body 12 must pass through the constrainer 11. The problem will now be described in more detail.

As widely known in the art, the size of any vertebral fixing device is limited to a great extent because it is a small device intended to be installed into the human body along with other devices, including a connecting rod 23 and a crosslink for interconnecting a pair of connection rods. However, although the overall size is limited in this manner, the anchor body 12, which is installed into a-vertebra, must have at least a predetermined dimension, particularly a thickness large enough to ensure that it provides a stable supporting force.

However, the fact that the anchor head 13 must engage with the constrainer 11 while the anchor body 12 must pass through the constrainer 11 in the case of the conventional vertebral fixing device inevitably limits the thickness of the anchor body 12. This is because any increase in thickness of the anchor body 12 is necessarily followed by the increase in size of the anchor head 13 and the constrainer 11, which is supposed to receive it, and thus enlarges the connecting rod 23 and the crosslink, which are associated with these components, to such an extent that they can hardly be installed into the body of a patient. Furthermore, relevant equipment e.g. equipment for disinfecting and cleaning the vertebral fixing device, must also be replaced.

In summary, even though the conventional vertebral fixing device has an advantage in that the constrainer 11 can be freely moved during surgery and conveniently connected to the connecting rod, the conventional device has a serious problem in that the diameter of the anchor body 12 cannot be increased without limitation, making it unsuited to certain types of surgery requiring a reliable supporting force.

### SUMMARY OF THE INVENTION

Accordingly, the present invention has been made to solve the above-mentioned problems occurring in the prior art. A vertebral fixing device of the present invention adapted a constrainer and pivot to freely swivel with regard to the anchor so that it can be easily coupled to a connecting rod, which has no limitation on the diameter of the anchor body.

In accordance with an aspect of the present invention, a vertebral fixing device is provided, which is comprising: a constrainer having a receiving cutouts formed on a first end to receive a connecting rod crosswise, and a inserting-mouth formed on a bottom; an anchor having a screw body to be coupled to a vertebra and a stem inserted into the constrainer via the inserting-mouth from the bottom side; a pivot adapted to enclose the annular ring and stem of the anchor after the annular ring has passed through the inserting-mouth so that the pivot is coupled to the anchor stem. The pivot is mounted on a peripheral seat of the inserting-mouth; and a constraint nut coupled to the constrainer housing by rotating to inward or outward so that when the constraint nut moves inward, the constraint nut firmly fasten the connecting rod and the pivot against the peripheral seat of the inserting-mouth, and when the constraint nut moves outward, the constraint nut releases the connecting rod and the pivot to allow the constrainer swivel with respect to the anchor.

The pivot has an insertion hole, the annular ring of the anchor being inserted into the insertion hole, and a plurality of slits extending from a mouth of the insertion hole, so that the insertion hole can be instantly expanded and shrunk.

An annular ring (124) of the anchor is snapped to fit into the snap-fitting groove (135) of the pivot, when the stem of the anchor is aligned to the inserting hole of the pivot and depressed, so that the annular ring of the anchor is snapped into fit the snap-fitting groove The annular ring (124) has a lateral surface (124A), which is gradually widened to have a slanted lateral surface, so that the snap-fitting annular ring (124) can be easily and firmly inserted into the insertion hole (131) of the pivot (130).

The peripheral seat of the inserting-mouth is formed a hemispherical surface, which the inner diameter is gradually decreasing outward along the thickness, and a lower portion of the pivot contacting the peripheral seat of the inserting mouth of the hemispherical shape so that the pivot is fastened to the annular ring and anchor stem as the pivot is pressed against.

The constrainer includes a constrainer base forming the inserting mouth and the peripheral seat, and constraint mount forming a first and second arms extending from the constrainer base, which is formed a pair of cutouts for receiving the connecting rod between the first and second arms.

A series of the female thread is formed on the inner surfaces of the first and second arms and a male thread has formed on the outer surface of the constraint nut to be coupled each other.

A constraint nut (140) having a male thread (141) formed on outer peripheral surface, and a hexagonal recess (143) on a head to be rotated by a tool, such as a hexagonal wrench. The constraint nut (140) is rotated clockwise to press the pivot, stem of the screw body and the connecting rod against the peripheral seat of the constraint housing. When the constraint nut (140) is rotated counterclockwise, the connecting rod and the pivot are released to swivel with respect to the anchor.

The stem of the anchor has smaller diameter than that of the screw body of the anchor.

### BRIEF DESCRIPTION OF THE DRAWINGS

FIG. 1 is a perspective view of a conventional vertebral fixing device.

FIG. 2 is a perspective view of a vertebral fixing device according to the present invention.

FIG. 3 is an exploded perspective view illustrating the construction of the vertebral fixing device according to the present invention.

FIG. 4 is a cross-section view of an assembled vertebral fixing device, which is cutting through the longitudinal direction according to the present invention.

FIG. 5 is a cross-section view showing the swivel operation of the vertebral fixing device with respect to the pivot according to the present invention.

FIG. 6 shows an application of the vertebral fixing device according to the present invention.

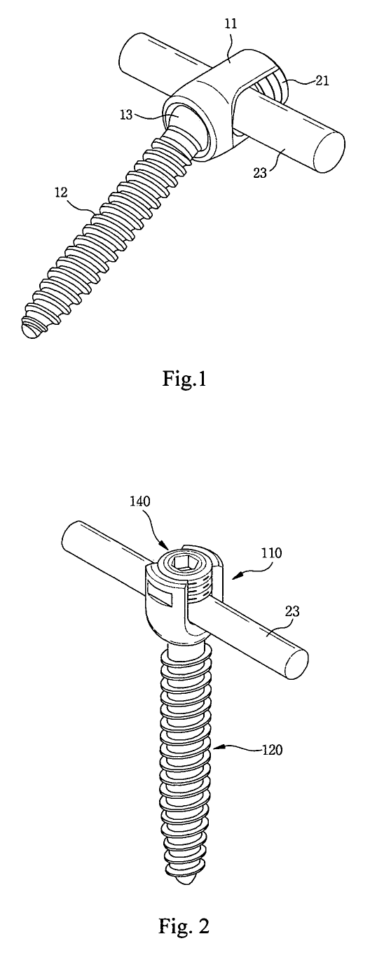

### DETAILED DESCRIPTION OF THE PREFERRED EMBODIMENT

Hereinafter, an exemplary embodiment of the present invention will be described with reference to the accompanying drawings. The identifying or similar components are used the same reference numerals in the following description and drawings. Thus, the repetition of the descriptions for the same or similar components will be avoided.

FIG. 2 is a perspective view of a vertebral fixing device according to the present invention. Although the vertebral fixing device of the present invention shown in FIG. 2 looks quite similar to the conventional vertebral fixing device, which has an anchor head thicker than the body so that the head engages with the constrainer while the body passes through the constrainer, a device of the instant invention is substantially different as follows: the stem of the anchor 120 is small enough to be passed through a pivot 130, and inserted into a constrainer nut 140. The pivot 130 and the constrainer nut 140, which are separated members, are coupled to the head of the anchor 120, so that they are inserted into the constraint housing 110. This construction has an advantage, since the screw body of the anchor 120 does not necessarily pass through the constraint housing 110, the header of the anchor 120 does not need to increase thickness for receiving it. In other words, the diameter of the anchor 120 can be increased to secure a reliable supporting force with regard to the corresponding vertebra without increasing the size of the head of the anchor 120 or the constraint housing 110.

The construction of the vertebral fixing device according to the present invention will now be described in detail.

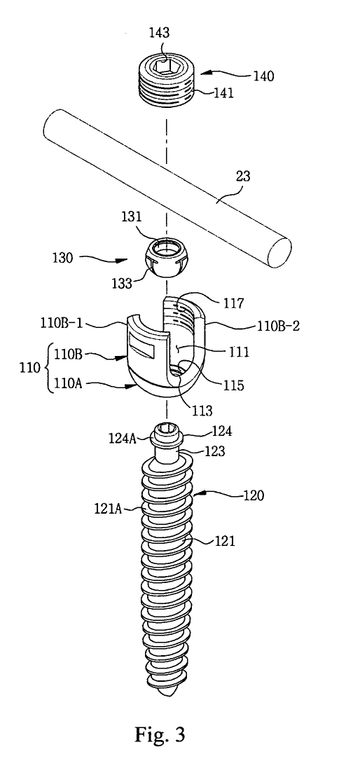

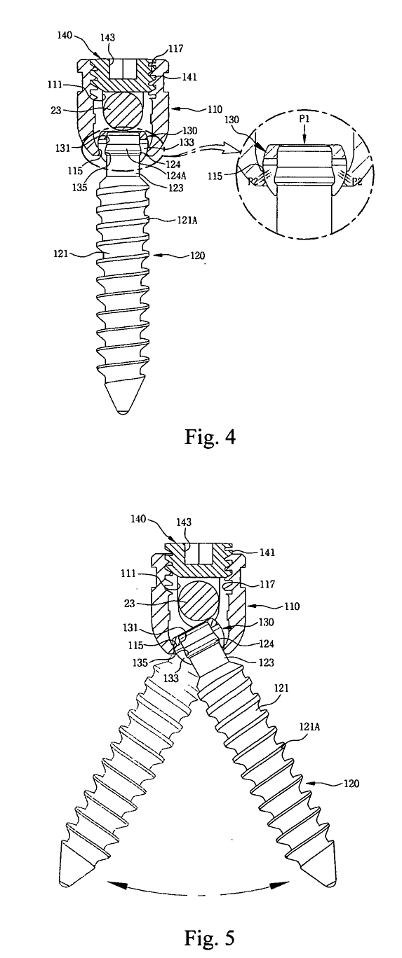

FIG. 3 is an exploded perspective view showing the construction of the vertebral fixing device according to the present invention; FIG. 4 is a longitudinal sectional view of the vertebral fixing device according to the present invention; and FIG. 5 is a sectional view showing the operation of the vertebral fixing device according to the present invention.

As shown, the vertebral fixing device according to the present invention includes a constraint housing 110, an anchor 120, a pivot 130, and a constraint nut 140.

The constraint housing 110 works in conjunction with the pivot 130 and the constraint nut 140 for coupling the anchor 120 and the connecting rod 23. The constraint housing 110 includes a constraint base 110A adapted to engage with the anchor stem 123 via the pivot 130, and a constraint mount 110B having two lateral cutouts 111 for receiving the connecting rod 23.

The constraint base 110A has an inserting-mouth 113 so that the anchor stem 123 can pass through from the bottom. The inserting-mouth 113 has a peripheral seat 115 for supporting the pivot 130. The peripheral seat 115 is formed as a slanted surface in the manner of the diameter of the through-hole gradually decreases outwardly along the thickness. This configuration guarantees the pivot 130 retained inside of the constraint housing 110, when the external force acts on the hemispherical pivot 130 and pushes it against the through-hole peripheral seat 115, so that the pivot 130 is constrained, compressed, and immobilized. As a result, the pivot 130 is fastened to the anchor stem 123 in a reliable manner. The external force, labeled P1, acting on the through-hole peripheral seat 115 is shown in FIG. 4, and the resultant force, labeled P2, acting between the pivot 130 and the through-hole peripheral seat 115 is also shown in FIG. 4.

The constraint mount 110B includes first and second arms 110B-1 and 110B-2, which are extended along the edge of the constraint base 110A disposed apart and faced each other. As a result, two lateral cutouts 111 are formed clearances between the first and second arms 110B-1 and 110B-2 to receive the connecting rod 23 crosswise. A series of threads 117 is formed on the inner surfaces of the first and second arms 110B-1 and 110B-2, so that the constraint nut 140 can be threaded into the constraint housing 110.

The anchor 120 includes an anchor screw 121 and an anchor stem 123. The anchor screw 121 is screwed into and fixed to a vertebra. The anchor stem 123 is inserted into the constraint housing 110 via the inserting-mouth 113 to be coupled to the pivot 130.

The anchor screw 121 has an external screw thread 121 A so that it can be drilled to be firmly fixed into a vertebra.

The anchor stem 123 is inserted via the inserting-mouth 113 of the constraint housing 110. The size of the anchor stem 123 is determined to provide a tolerance when it is inserted via the inserting-mouth 113 of the constraint housing 110. This is for the purpose of ensuring that, when the anchor stem 123 has been inserted into the constraint housing 110, the constraint housing 110 can swivel freely (e.g., roll, tilt). The anchor stem 123 has a snap fitting annular ring 124 around the outer peripheral surface of the anchor stem 123. The snap-fitting annular ring 124 is coupled to the corresponding snap-fitting groove 135 formed in the insertion hole 131 of the pivot 130. In other words, the snap-fit annular ring 124 on the anchor stem 123 simply snaps into the snap-fitting grooves 135 of the pivot 130. The snap-fitting annular ring 124 has a lateral surface 124A, which is gradually widened to have a slanted lateral surface, so that the snap-fitting annular ring 124 can be easily and firmly inserted into the insertion hole 131 of the pivot 130.

The constraint base 110A is adapted a peripheral seat 115 around the inserting-mouth 113 to mount the pivot 130, while it is enclosing and coupling to the anchor stem 123, which has passed through the inserting-mouth 113 of the constraint housing 110. Thus, the pivot 130 prevents the anchor stem 123 escaping from the constraint housing 110. The pivot 130 has an insertion hole 131 for inserting the stem of the anchor 123. The pivot 130 has a plurality of slits 133 extending from the mouth of the insertion hole 131, so that the insertion hole 131 can instantly expanded and shrank when the annular ring 124 and the anchor stem 123 is inserted. The pivot 130 has a snap-fitting grooves 135 formed on the inner peripheral surface of the insertion hole 131 to be coupled with the corresponding snap-fitting annular ring 124 of the anchor stem 123. The pivot 130 has a hemispherical shape, i.e., the portion of the pivot 130 contacting on the through-hole peripheral seat 115 of the constraint housing 110 forms a curved surface.

This configuration of the pivot 130 guarantees that the anchor stem 123 simply snaps into the insertion hole 131 of the pivot 130 when the anchor stem 123 is inserted into the constraint housing 110 via the inserting-mouth 113. If a force is applied from the constraint nut 140 after the pivot 130 and the anchor stem 123 have coupled each other, the pivot 130 is forced against the curved through-hole peripheral seat 115 to be constrained and compressed. Further, the constraint housing with the anchor stem 123 is reinforced.

The constraint nut 140 has a male thread 141 formed on its outer peripheral surface so that the constraint nut 140 can be threaded into the first and second arms 110B-1 and 110B-2 of the constraint housing 110. The constraint nut 140 can be threaded inward or outward for coupling or releasing by rotating the constraint nut 140 counter clockwise or clockwise. The connecting rod 23, the pivot 130 and the anchor stem 123 can be pressed against the through-hole peripheral seat 115. As a result, the pivot 130 is compressed against the through-hole peripheral seat 115, which is formed as a curved surface, to be constrained and immobilized. The pivot 130 is also shrunk and further reinforced the constraint housing with the anchor stem 123. The connecting rod 23 is also strongly fastened and constrained between the constraint nut 140 and the pivot 130. If the constraint nut 140 is rotated in the opposite direction and threaded outward, the anchor stem 123 and the constraint housing 110 are decompressed and released as shown in FIG. 5. Thus, they are released with regard to each other. This operation is also released the connecting rod 23. The constraint nut 140 has a hexagonal recess 143 formed on the head, so that it can be rotated by a tool such as a hexagonal wrench.

The operation and function of the vertebral fixing device according to the present invention, which has the above-mentioned construction, will now be described in detail with reference to the accompanying drawings.

First, the anchor 120 and the constraint housing 110 are assembled by means of the pivot 130. Particularly, the anchor stem 123 is inserted into the constraint housing 110 via the inserting-mouth 113 from the bottom.

The pivot 130 is then coupled to the anchor stem 123, which is inserted into the constraint housing 110. Especially, the pivot 130 is aligned to the anchor stem 123, so that the anchor stem 123 is inserted into the insertion hole 131 of the pivot 130.

Then, the anchor stem 123 is pushed into the insertion hole 131 of the pivot 130 until the snap-fitting annular ring 124 snaps into the snap-fitting groove 135. During the inserting process, the slits 133 is monetarily expanded to be widen the mouth of the insertion hole 131 of the pivot 130, so that the anchor stem 123 can be smoothly inserted through the insertion hole 131. As soon as the snap-fitting annular ring 124 snaps into the snap groove 135, the insertion hole 131 will be shrunk to the original shape. After the anchor stem 123 and the pivot 130 are coupled in this manner, the pivot 130 is mounted on the through-hole peripheral seat 115 of the constraint housing 110 and retained the anchor stem 123.

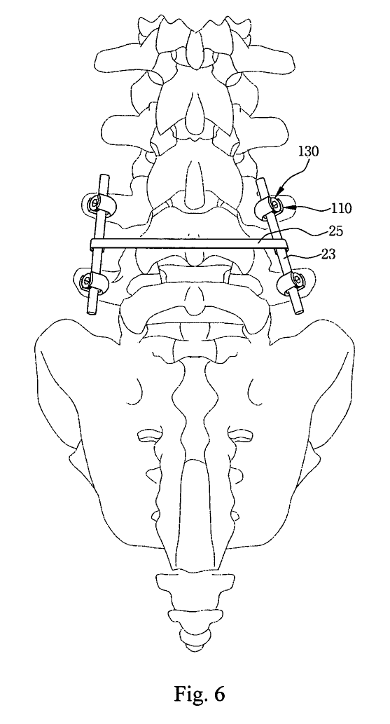

This is followed by a process of affixing the assembly of the constraint housing 110 and the anchor 120, which have been coupled to each other, to a vertebra as shown in FIG. 6. Especially, the sharp end point of the anchor screw 121 is pressed against a hard portion of a vertebra, such as a pedicle, or a sacrum to be rotated by a tool. Then, the anchor screw 121 having a screw thread 121A, is drilled into and firmly affixed into the vertebra. The primary process completes to affix the anchor 120 assembled with the constraint housing 110 onto the vertebra. First, the anchor 120 and the constraint housing 110 are coupled to each other, then the assembly is screwed into a vertebra. It is also possible to firmly insert the anchor 120 into a vertebra and then the constraint housing 110 coupled to the anchor 120.

As described above, a number of assembled constrainers 110 and the anchors 120 are installed on the respective vertebrae. A connecting rod 23 is aligned to connect the assembled constrainers 110 and the anchors 120. Particularly, the connecting rod 23 is inserted through the lateral cutouts 111 of each constraint housing 110. The insertion process is easily performed due to the swiveling of the constraint housing 110 with respect to the pivot 130 with the anchor stem 123.

The connecting rod 23 is then fastened to the constraint housing 110. Particularly, a constraint nut 140 is inserted into the first and second arms 110B-1 and 110B-2 of a constraint housing 110 for rotation. The constraint nut 140 is threaded into the threads 141 and 117 of the first and second arms 110B-1 and 110B-2, respectively. The constraint nut 140 is further rotated in the clockwise direction, so that the constraint nut 140 advanced inward along the inner thread of the first and second arms 110B-1 and 110B-2 and presses the connecting rod 23 and the pivot 130 inserted through the two lateral cutouts 111. As a result, the hemispherical pivot 130 is firmly pressed against the through-hole peripheral seat 115, which has formed a curved surface, to be constrained and immobilized. The pivot 130 also contracts and further reinforces the constraint housing along with the anchor stem 123. The connecting rod 23 is also pressed between the constraint nut 140 and the anchor stem 123 to be constrained and immobilized. The process as described above is repeated to install the second constraint housing 110 for aligning and installing the connecting rod 23.

As shown in FIG. 6, the installation of the assembled constrainers 110 and the anchors 120 and the connecting rods 23 are performed on the left and right, and install a crosslink 25. The process is completed to fix the vertebral by using the device of the present invention for a surgery

As mentioned above, the vertebral fixing device according to the present invention has a unique feature, i.e. a separate pivot adapted to engage with the constrainer while enclosing the anchor head, so that the anchor body can have a sufficient thickness while ensuring free movement of the constrainer with regard to the anchor during surgery.

The pivot and the annular ring of the anchor stem are simply snapped onto each other so that any inconvenient or complicated structure for coupling the pivot and the anchor head is unnecessary.

The insertion hole formed on the pivot can instantly expand and shrink due to the slits formed at its mouth, so that the anchor stem can be easily inserted into and coupled to the pivot.

Further, the anchor stem of the present invention can make smaller diameter than that of the anchor body.

Although an exemplary embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A device for fixing vertebral comprising;
a constraint housing (110) having a constraint base (110A) and a constraint mount (110B) formed two lateral cutouts (111) for receiving a connecting rod (23),
an anchor (120) having an anchor screw (121) for installing onto the vertebral, an anchor stem (123) be inserted into the constraint housing (110), and an annular ring (124),
a pivot (130) having an insertion hole (131), a plurality of slits (133) extended from a mouth of the insertion hole (131), a snap-fitting groove (135) formed on the inner peripheral surface to be snap-fitting to the annular ring (124),
a constraint nut (140) having a male thread (141) formed on outer peripheral surface, and a hexagonal recess (143) on a head to be rotated by a tool, such as a hexagonal wrench, wherein the constraint nut (140) is rotated clockwise to press the pivot, stem of the screw body and the connecting rod against the peripheral seat of the constraint housing and when the constraint nut (140) is rotated counterclockwise, the connecting rod and the pivot are released to swivel with respect to the anchor.

2. The device for fixing vertebral as claimed in claim 1, wherein said constraint base (110A) of the pivot is further comprising an inserting mouth (113) at bottom, for inserting the stem of the anchor.

3. The device for fixing vertebral as claimed in claim 1, wherein said annular ring (124) of the anchor is snapped to fit into the snap-fitting groove (135) of the pivot, when the stem of the anchor is aligned to the inserting hole of the pivot and depressed, so that the annular ring of the anchor is snapped into fit the snap-fitting groove.

4. The device for fixing vertebral as claimed in claim 3, wherein said annular ring (124) has a lateral surface (124A), which is gradually widened to have a slanted lateral surface for easily snapping-fit, so that the snap-fitting annular ring (124) can be easily and firmly inserted into the insertion hole (131) of the pivot (130).

5. The device for fixing vertebral as claimed in claim 2, wherein said inserting mouth (113) further comprising a peripheral seat (115) for supporting the pivot (130), wherein said peripheral seat (115) is formed a hemi-spherical surface, which the diameter of the inserting-hole is outwardly and gradually decreases along the thickness direction.

6. The device for fixing vertebral as claimed in claim 1, wherein said constraint base (110A) further comprising a peripheral seat (115) and an inserting-mouth (113) for inserting through the annular ring (124) and the stem (123) of the anchor (121) from bottom, said constraint mount (110B) forming first and second arms (110B-1, 110B-2) being extended along the edge of the constraint base (110A) and disposed apart to be faced each other.

7. The device for fixing vertebral as claimed in claim 6, wherein said first and second arms (110B-1, 110B-2) form a series of inner thread to be coupled to the outer thread of the constraint nut.

8. The device for fixing vertebral as claimed in claim 1, wherein the stem of the anchor has a diameter smaller than that of the body of the anchor.
